(19) United States
(12) Patent Application Publication
(10) Pub. No.: US 2009/0105632 A1
(43) Pub. Date: Apr. 23, 2009

(54) Electrotransport Of Lisuride

(76) Inventors: Rama V. Padmanabhan, Los Altos, CA (US); Joseph B. Phipps, Sunnyvale, CA (US); Janardhanan Anand Subramony, Belle Mead, NJ (US); Stephen S. Hwang, Palo Alto, CA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/246,856

(22) Filed: Oct. 7, 2008

Related U.S. Application Data
(60) Provisional application No. 60/980,847, filed on Oct. 18, 2007.

(51) Int. Cl. A61K 31/437; A61N 1/30; H01S 4/00; A61P 25/16

(52) U.S. Cl. 604/20; 604/501; 514/288; 29/592.1

(57) ABSTRACT

An electrotransport system for delivery of lisuride or a pharmaceutically acceptable salt thereof. The system has a donor electrode assembly, a counter electrode assembly, and a controller electrically connected to the donor electrode assembly and the counter electrode assembly. The donor electrode assembly has a donor reservoir that contains a lisuride salt. The controller is electrically connected to the donor and counter electrode assemblies and is operatable for controlling current for the electrotransport.

### CROSS REFERENCE TO RELATED APPLICATIONS

This application claims the benefit of U.S. patent application No. 60/980,847, filed Oct. 18, 2007, which is incorporated herein by reference in its entirety.

### TECHNICAL FIELD

The present invention relates to an electrotransport drug delivery system and method for the electrotransport delivery of lisuride, or pharmaceutically acceptable salts thereof, to patients in need of treatment with lisuride. The invention further relates to methods of treating Parkinson's disease that involve delivery via electrotransport of lisuride, or pharmaceutically acceptable salts thereof to patients in need of such treatment.

### BACKGROUND

The delivery of active agents through the skin provides many advantages, including comfort, convenience, and non-invasiveness. Gastrointestinal irritation and the variable rates of absorption and metabolism including first pass effect encountered in oral delivery are avoided. Transdermal delivery also provides a high degree of control over blood concentrations of any particular active agent.

Many active agents are not suitable for passive transdermal delivery because of their size, ionic charge characteristics, and hydrophilicity. Body surfaces such as skin are very effective barrier against intrusion by drugs or chemical agents. Generally hydrophilic agents do not permeate passively through body surface such as skin well because of the lipid-containing cellular structure of skin. One method for transdermal delivery of such active agents involves the use of electrical current to transport actively the active agent into the body through a body surface (e.g., intact skin) by electrotransport, which differs from passive delivery in that additional energy is used to drive the movement of the active agent. Electrotransport techniques may include iontophoresis, electroosmosis, and electroporation. Electrotransport devices, such as iontophoretic devices are known in the art, e.g., U.S. Pat. No. 5,057,072; 5,084,008; 5,147,297; 5,395,310; 5,503,632; 5,871,461; 6,039,977; 6,049,733; 6,181,963, 6,216,033, 6,881,208, and US Patent Publications 20020128591, 20030191946, 20060089591, 20060173401, 20060241548. One electrode, called the active or donor electrode, is the electrode from which the active agent is delivered into the body. The other electrode, called the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the ionic substance to be driven into the body is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode. A prior iontophoretic drug delivery system for the delivery of fentanyl is the IONSYS™ system (Janssen-Cilag). Characteristics of the IONSYS™ system can be found in Annex I (Summary of Product Characteristics) of the Product Information (Jan. 24, 2006 Ionsys-H-C-612-00-00) of IONSYS™ system, available from the European Medicines Agency (EMEA).

Lisuride is a compound the free base form of which is known as N′-[(8α)-9,10-didehydro-6-methylergolin-8-yl]-N,N-diethylurea and as 1,1-Diethyl-3-((6aR,9S)-7-methyl-4,6,6a,7,8,9-hexahydroindolo[4,3-fg]quinolin-9-yl)urea. The following is a representation of the structure of lisuride free base, with a formula of C₂₀H₂₆N₄O and a molecular weight of about 338.45.

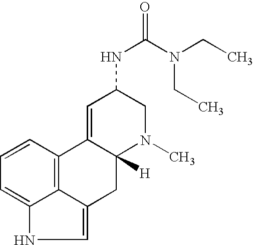

Lisuride is also commonly available as a hydrogenmaleate salt. When taken in the oral route, it is metablized by the liver and the bioavailability is low (about 25%). Lisuride is a water soluble molecule with a pKa of about 8 and has a +1 charge in the acidic range all the way to about pH7. The charge falls to zero about pH9. Lisuride is somewhat soluble in water in the acid range of 4-6. Its solubility is about 1.8 mg/ml in 10 mM citrate buffer at pH 5. Lisuride has been known to produce benefit for migraine and has been used for treating Parkinson's disease.

Parkinson's disease is a chronic movement disorder and is progressive. There are about 1 million patients in the US and about 4 million patients worldwide. The symptoms of the disease continue and worsen over time. Parkinson's disease occurs when a group of cells in an area of the brain called the substantia nigra begin to malfunction and die. These cells normally produce a neurotransmitter dopamine, which controls movement and coordination. In a person with Parkinson's disease, because of the death of dopamine-producing cells the amount of dopamine produced in the brain decreases, resulting in difficulties in controlling movements. Symptoms commonly seen in patients with Parkinson's disease include tremor of the hands and limbs, rigidity or stiffness, and postural instability. Other symptoms also include difficulty in swallowing, chewing, and speaking; urinary problems or constipation; skin problems; and sleep disruptions. As the disease progresses and worsen, the shaking, or tremor, difficulty in swallowing and chewing can interfere with daily activities.

At present, there is no cure for Parkinson's disease. Generally medication is used for relief from the symptoms. Typically, for relief, patients are given levodopa (L-dopa) combined with carbidopa (C-dopa). Carbidopa delays the conversion of levodopa into dopamine until it reaches the brain. Nerve cells can use levodopa to make dopamine.

Although levodopa helps many of the patients in relieving some of the symptoms, in many cases tremors continue. Further, with time, patients may tend to respond less to levodopa, as the drug tends to lose its effectiveness after the first two to three years of treatment. After five to six years, only 25% to 50% of patients on L-dopa therapy maintain improvement. Another undesirable effect of Parkinson's disease under current therapies is the occurrence of the “fluctuation syndrome,” in which the patient experiences alternating “on” periods of mobility with dyskinesias and “off” periods with hypokinesia or akinesia. Continuous infusions of dopamine receptor agonist may help ease symptoms of the “fluctuation syndrome”.

Eventually, when a patient does not respond to medication, surgery may be used as a last resort to alleviate symptoms. Thus, there is an urgent need for medication that can replace or supplement levodopa in the treatment of Parkinson's disease. As mentioned above, lisuride has been used successfully in relieving symptoms of Parkinson's disease. In oral coadministration with levodopa, lisuride has been shown to be an effective in oral doses of about 1 mg/day (see, H. Allain et al., European Neurology, 2000;44:22-30). Lisuride has been shown to be an effective in treating Parkinson's disease in oral doses of about 1.6 to 5.0 mg/day with a mean of 3.6 mg/day if used alone in monotherapy (see, Abraham Lieberman, et al., NEUROLOGY 1981;31:961). In many published reports, lisuride is given at doses ranging from 1.5 to 4.5 mg/day (see also Movement Disorders, Vol. 17, Suppl. 4, 2002, p. S74-S78).

However, oral administration of lisuride may sometimes be undesirable. First, oral bioavailability is low. Second, the present oral doses may lead to fluctuations of plasma drug levels. There is therefore a continuing need for non-oral administration of lisuride, especially in a sustained release platform. The present invention delivers lisuride by electrotransport for therapeutic benefits in patients with Parkinson's disease.

### SUMMARY

The present invention relates to electrotransport systems and methods of transdermal delivery of lisuride or a pharmaceutically acceptable salt thereof, and method of making such systems.

In one aspect, a device for the transdermal electrotransport of lisuride to a patient in need thereof for therapeutic benefit is provided. The system has a donor electrode assembly, a counter electrode assembly, and a controller electrically connected to the donor electrode assembly and the counter electrode assembly. The donor electrode assembly has a donor reservoir that contains a lisuride salt. The controller is electrically connected to the donor and counter electrode assemblies and is adapted to be used for controlling current for the electrotransport.

In another aspect, a method for the transdermal electrotransport of lisuride or a pharmaceutically acceptable salt thereof to a patient in need thereof for therapeutic benefits is provided. The method includes placing a device for the electrotransport delivery of lisuride on a patient and using the device to administer lisuride by electrotransport. The device used in the method has a donor electrode assembly, a counter electrode assembly; and a controller electrically connected to the donor and counter electrode assemblies to control current flow. The donor electrode assembly has a donor reservoir containing lisuride salt. Lisuride salt is a source for lisuride cation that can be delivered by electrical potential difference in electrotransport through the body surface so that lisuride can be delivered into the systemic circulation.

In the present invention, it has been discovered using the electrotransport device (e.g., by iontophoretic delivery) that we can deliver lisuride to a patient in amount at a rate that has been shown to be therapeutically effective via oral administration by prior work Since transdermal delivery is a route that would render better bioavailability compared to oral delivery due to the low oral bioavailability of lisuride, the present invention will be able to provides therapeutic benefits to patients suffering from Parkinson's disease.

In another aspect of the invention, by using an electrotransport device that has lisuride hydrochloride in the donor reservoir and using silver electrode in the anode, and further including anion exchanger resin in chloride form in the donor reservoir, it is practicable to deliver lisuride by electrotransport at a therapeutically effective rate for at least 12 hours without resulting in silver staining on the body surface, e.g., skin. During electrotransport, chloride ions precipitate out metal (e.g., silver) ions generated at the metal (silver) in the anode. Yet, lisuride hydrochloride need not be included in large excess to provide the chloride necessary for metal ion precipitation because the ion exchanger also provides much of the chloride ions to precipitate the competing metal ions, preventing skin discoloration caused by, e.g., silver staining.

Thus, with the present invention, one will be able to deliver lisuride or a pharmaceutically acceptable salt thereof for therapeutic benefit to a patient. Delivery can be done, for example, at a flux of at least 8 microgram/(cm²hr), i.e., μg/(cm²hr), of lisuride base equivalent using a current of 50 microA/cm², i.e., 50 μA/(cm²hr) or more. Such lisuride delivery can be done without metal staining of the body surface such as skin. Because the delivery rate of lisuride by electrotransport is comparable to delivery rate needed for treatment of Parkinson's disease, lisuride delivery as disclosed herein is effective for treating Parkinson' disease. Further, since lisuride has been used in the treatment of migraine, lisuride by electrotransport as disclosed herein can also be used for treating migraine.

### BRIEF DESCRIPTION OF THE FIGURES

The present invention is illustrated by way of examples in embodiments and not limitation in the figures of the accompanying drawings in which like references indicate similar elements. The figures are not shown to scale unless indicated otherwise.

FIG. 1 illustrates an exploded, isometric view of an embodiment of a typical electrotransport system similar to a lisuride delivery device of this invention.

FIG. 2 is a graphical representation of the flux of lisuride in lisuride base equivalent by electrotransport done on skin samples from 3 donors in accordance with this invention.

### DETAILED DESCRIPTION

The present invention is related to electrotransport delivery systems and methods for transdermally delivering lisuride, which is useful in treating Parkinson's disease. The system can be applied to deliver drug to a body surface (e.g., transdermally through skin) into the systemic circulation.

The practice of the present invention will employ, unless otherwise indicated, conventional methods used by those skilled in the art in pharmaceutical product development.

In describing the present invention, the following terminology will be used in accordance with the definitions set out below.

The singular forms “a,” “an” and “the” include plural referents unless the context clearly dictates otherwise. Thus, for example, reference to “a polymer” includes a single polymer as well as a mixture of two or more different polymers.

As used herein, the terms “electrotransport,” “iontophoresis,” and “iontophoretic” refer to the delivery of pharmaceutically active agents (charged, uncharged, or mixtures thereof) through a body surface (such as skin, mucosal membrane, eye, or nail) wherein the delivery is at least partially induced or aided by the application of an electric potential. The agent may be delivered by electromigration, electroporation, electroosmosis or any combination thereof. Electromigration (also called iontophoresis) involves the electrically induced transport of charged ions through a body surface by electrical potential difference. Electroosmosis has also been referred to as electrohydrokinesis, electro-convection, and electrically induced osmosis. In general, electroosmosis of a species into a tissue results from the migration of solvent in which the species is contained, as a result of the application of electromotive force to the therapeutic species reservoir, i.e., solvent flow induced by electromigration of other ionic species. During the electrotransport process, certain modifications or alterations of the skin may occur such as the formation of transiently existing pores in the skin, also referred to as “electroporation.” Any electrically assisted transport of species enhanced by modifications or alterations to the body surface (e.g., formation of pores in the skin) are also included in the term “electrotransport” as used herein. Thus, as used herein, the terms “electrotransport,” “iontophoresis” and “iontophoretic” refer to (1) the delivery of charged drugs or agents by electromigration, (2) the delivery of uncharged drugs or agents by the process of electroosmosis, (3) the delivery of charged or uncharged drugs by electroporation, (4) the delivery of charged drugs or agents by the combined processes of electromigration and electroosmosis, and/or (5) the delivery of a mixture of charged and uncharged drugs or agents by the combined processes of electromigration and electroosmosis. The present invention is especially applicable for electromigration iontophoretic drug delivery.

As used herein, unless specified to be otherwise in content, “distal” refers to a direction pointing away or being more distant to the body surface, “proximal” refers to a direction pointing to or being nearer to the body surface.

The terms “drug” and “therapeutic agent” mean any therapeutically active substance that is delivered to a living organism to produce a desired, usually beneficial, effect, such as relief of symptoms or discomfort, treatment of disease, or adjustment of physiological functions, e.g., analgesic, regulation of hormone, antimicrobial action, sedatives, etc.

As used herein, the term “lisuride” generally refers to lisuride free base and/or lisuride salt unless specified to the otherwise or the context of its use is clear that it is meant to be otherwise. All flux or dose values are in lisuride base equivalent unless specified to be otherwise.

As used herein, the term “matrix” when relates to a drug reservoir refers to a porous, composite, solid, or semi-solid substance, such as, for example, a polymeric material or a gel, that has pores or spaces sufficiently large for lisuride or a pharmaceutically acceptable salt thereof to populate. The matrix serves as a repository in which lisuride or its pharmaceutically acceptable salt is contained.

The term “pharmaceutically acceptable salt” refers to salts of lisuride that retain the biological effectiveness and properties of lisuride, and that are not biologically or otherwise undesirable.

The term “salt” means a compound in which the hydrogen of an acid is replaced by a metal or its equivalent. As used herein, the salt can be in ionized in solution or in undissociated form (e.g., in solid form).

As used herein, the terms “transdermal administration” and “transdermally administering” refer to the delivery of a substance or agent by passage into and through the skin, mucous membrane, the eye, or other surface of the body into the systemic circulation.

The term “treatment” a disease means the application of medication, surgery or other procedure for relieving symptoms or curing the disease from a patient.

The term “therapeutic benefit dose” or “therapeutic effective dose” refers to the amount of a drug, e.g., lisuride or a pharmaceutically acceptable salt thereof that, when administered to a patient, is effective to at least partially treat a condition from which the patient suffers, e.g., with the coadministration of levodopa in treating Parkinson's disease. The therapeutic benefit can be the control or reduction of symptoms, such as tremors, etc., of the patient, known to be associated with Parkinson's disease.

### MODES OF CARRYING OUT THE INVENTION

The present invention provides an anode for electrotransport delivery of lisuride, or a pharmaceutically acceptable salt thereof, through a body surface, such as skin or mucosal membrane, e.g., buccal, rectal, etc.

Electrotransport devices, such as iontophoretic devices are known in the art, e.g., U.S. Pat. No. 5,503,632, U.S. Pat. No. 6,216,033, US20060089591, can be adapted to include lisuride or a pharmaceutically acceptable salt therefore for use for the treatment of Parkinson's disease. The electrotransport drug delivery system typically includes portions having a reservoir associated with either an anodic electrode or a cathodic electrode (“electrode/reservoir portions”). Generally, both anodic and cathodic portions are present. The electrode/reservoir portion is for delivering an ionic drug or counter ions. The electrode/reservoir portion for the drug reservoir typically includes a drug reservoir in layer form that is to be disposed proximate to or on the skin of a user for delivery of drug to the user. The reservoir can be a matrix that can hold a drug in liquid form, e.g., solution. The drug reservoir typically includes an ionic or ionizable drug. In the case of lisuride, the lisuride is in the anodic reservoir. The typical iontophoretic transdermal device can have an activation switch in the form of a push button switch and a display in the form of a light emitting diode (LED) or an alpha-numeric display (e.g., LCD) as well. Electronic circuitry in the device provides a means for controlling current or voltage to deliver the drug via activation of the electrical delivery mechanism. The electronics are housed in a housing and an adhesive typically is present on the housing to attach the device on a body surface, e.g., skin, of a patient such that the device can be worn for many days, e.g., 1 day, 2 days, 3 days, etc.

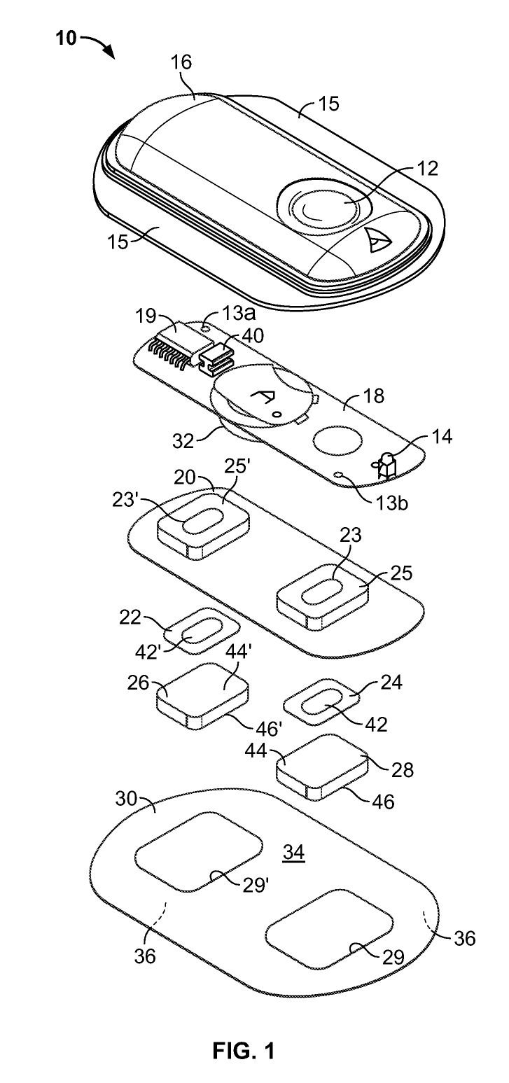

A prior iontophoretic system similar to that of U.S. Pat. No. 6,181,963 is shown in FIG. 1. FIG. 1 shows a perspective exploded view of an electrotransport device 10 having an activation switch in the form of a push button switch 12 and a display in the form of a light emitting diode (LED) 14. Device 10 includes an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable polymer.

Printed circuit board (PCB) assembly 18 includes an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of printed circuit board assembly 18 is a battery 32, preferably a button cell battery and most preferably a lithium cell. Other types of batteries may also be employed to power device 10. The circuit outputs (not shown in FIG. 1) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′, 29 in adhesive 30. The skin-facing side 36 of the adhesive 30 has adequate adhesive property to maintain the device on the skin for the duration of the use of the device.

The device for use for delivering lisuride or a salt thereof, can be similar to that shown in FIG. 1. The control system, associated with the printed circuit board can be designed in such a way the current and voltage can be controlled for its amplitude, duration, pulsation, wave shape, duty cycles, etc. Methods of designing, fabricating PCB and programming for such implementation is known to those skilled in the art.

The reservoir of the electrotransport delivery devices typically contains a gel matrix (although other non-gel reservoirs, such as spongy or fibrous pads holding liquid, and membrane confined reservoirs, can also be used instead), with the drug solution uniformly dispersed in at least one of the reservoirs. Gel reservoirs and methods for making and using such are described, e.g., in U.S. Pat. Nos. 6,039,977 and 6,181,963. Suitable polymers for the gel matrix can contain essentially any nonionic synthetic and/or naturally occurring polymeric materials. A polar nature is preferred when the active agent is polar and/or capable of ionization, so as to enhance agent solubility. Optionally, the gel matrix can be water swellable. Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent. It is to be understood that the application of the anodes and devices of the present invention is not limited by the reservoir carrier material so long as the reservoir can function to dissociate cationic drug and allow ions to migrate therein. For example, a reservoir that has a semiporous membrane containing a liquid, or a porous pad holding liquid are also applicable for use with an anodic electrode of the present invention.

In certain embodiments of the invention, the reservoir of the electrotransport delivery system comprises a polyvinyl alcohol hydrogel. The use of polyvinyl alcohol and method of making polyvinyl alcohol hydrogels are known in the art, for example, in U.S. Pat. No. 6,039,977. The weight percentage of the polyvinyl alcohol used to prepare gel matrices for the reservoirs of the electrotransport delivery devices, in certain embodiments of the methods of the present invention, is about 10 wt % to about 30 wt %, preferably about 15 wt % to about 25 wt %, and more preferably about 19 wt %. Preferably, for ease of processing and application, the gel matrix has a viscosity of from about 1,000 to about 200,000 poise, preferably from about 5,000 to about 50,000 poise.

To make hydrogels from polyvinyl alcohol (PVOH), polyvinyl alcohol is typically dissolved first (e.g., at 19 wt % in purified water at 90° C. for 30 minutes). Especially useful are the PVOH grades that are well hydrolyzed, e.g., 98 mol % or more hydrolyzed. For example, polyvinyl alcohol MOWIOL 28-99, which has 99 to 99.8 mol % hydrolysis or MOWIOL 10-98, which has 98 to 98.8 mol % hydrolysis (available from KURARAY) can be used. The gel solution is then dispensed into molds, and freezing overnight at about −20° C. For example, a useful PVOH solution can have a viscosity of 28 MPa·s (for a 4% aqueous solution at 20° C.). The formed hydrogel is then allowed to imbibe lisuride as a concentrated aqueous solution at room temperature to obtain the desired lisuride loading. Alternatively, lisuride loading is done by adding lisuride to the PVOH hydrogel solution before freezing. In the thermally processed formulations, PVOH can be dissolved in purified water at 90° C. as described above. After reduction of the temperature to 50° C., an aqueous solution of lisuride is added to the PVOH solution and allowed to mix for 30 minutes. The PVOH-lisuride mixture is dispensed into molds and freeze-cured. Finished hydrogels are used in flux studies, stability analysis, etc. Similarly, other forms of reservoir, made with a material different from PVOH can similarly be made by forming the reservoir with the drug or imbibing the drug into a form reservoir by one skilled in the art.

As in prior systems of electrotransport, one mode for delivery lisuride or its salt by electrotransport is the use of direct current. During iontophoretic electrotransport a current drives lisuride cations transdermally. Typically, the flux and therefore the amount of drug ions delivered as a function of time is proportional to the current. The use of a constant direct current signal typically provides a linear relationship between the applied current density and the flux of lisuride or its salt. Alternative electrotransport conditions can be employed, however. For example, pulsed current, alternating reverse polarity or time-varying, on-off current patterns may be suitable to prevent or minimize skin irritation if prolonged direct current delivery at a single location is undesirable. The electrotransport delivery devices of embodiments of the invention can utilize any suitable electrical circuits to perform a number of functions. Such circuits include pulsing circuits for delivering a pulsed current, timing circuits for delivering lisuride or a pharmaceutically acceptable lisuride salt over predetermined timing and dosing regimens, feedback regulating circuits for delivering lisuride or a pharmaceutically acceptable lisuride salt in response to a sensed physical parameter, and polarity controlling circuits for periodically reversing the polarity of the electrodes. Examples of iontophoretic delivery using pulsed current are U.S. Pat. No. 6,219,576 and U.S. Pat. No. 7,136,698. An example in which polarity is reversed is U.S. Pat. No. 4,406,658.

In the delivery of lisuride by a continuous direct current (DC), generally a current of about 20 to 500 μA/cm², preferably about 50 to 200 μA/cm², more preferably about 50 to 100 μA/cm², is used for good flux and minimized irritation to the skin. The size, including surface area, of the reservoir in contact with the body surface is designed to provide the flux for the duration of use. Generally, the surface area of the donor reservoir in contact with the body surface (donor reservoir contact area) can be about 0.5 cm² to 50 cm², preferably about 1 cm² to 10 cm². Generally the thickness of reservoir is about 0.1 mm to 5 mm, preferably about 0.5 mm to 1.5 mm. The lisuride-base-equivalent flux can be generally about 5 μg/(cm²hr) to 50 μg/(cm²hr), preferably about 8 μg/(cm²hr) to 25 μg/(cm²hr) based on the donor reservoir contact area. This is practicable with a direct current (DC) delivery of about 50 μA/cm² current density. With increased current, e.g., 100 μA/cm², the flux can be increase, e.g., doubled. Even higher current densities, e.g., 250 μA/cm², 500 μA/cm², can be used. Thus for lisuride electrotransport, the lisuride-base-equivalent flux is generally about 5 μg/(cm²hr) to 100 μg/(cm²hr), preferably about 8 μg/(cm²hr) to 50 μg/(cm²hr), more preferably about 8 μg/(cm²hr) to 25 μg/(cm²hr). Of course, a lower flux of, e.g., 5 to 15 μg/(cm²hr) can also be used to deliver a lower dose to patients who may not need a high dose of lisuride. The current density, and reservoir size can be chosen to deliver a larger dose or a smaller dose.

In the use of pulsed current, the duty cycle of the pulsed current delivery can be varied to result in the desired profile of drug delivery. Duty cycle is the ratio of the “on” time interval to the period of time of one cycle in the pulses (i.e., the ratio of the pulse-duration time to the pulse-period). The current pattern can be adjusted by changing the magnitude of the pulses or by changing the duty cycle of the pulses. Thus, the flux can be changed by increasing the magnitude of the current pulses or by increasing the duty cycle. For example, doubling the duty cycle or the magnitude of the current can double the flux. Further, the shape of the pulses can also be manipulated to control the drug delivery.

Another suitable type of electrotransport delivery may be characterized as alternating reverse polarity. An example of such a system is described in U.S. Pat. No. 4,406,658.

The electrotransport devices and methods of the invention can also be used in a feedback manner to create a closed loop. Physiological parameters can be monitored to provide feedback to the electrotransport device for control of the delivery. For example, a sensor can sense and send feedback based on abnormalities in motor fluctuations to the control circuitry to affect the dosing regimen. An example is the involuntary muscle contractions can be monitored using a voltammetric sensor and signals can be monitored at frequencies consistent with dyskinesias. Other sensors such as motion sensors and accelerometers can be used to implement the feedback control loop. Information from such monitoring is fed back to the control system in the electrotransport device to automatically adjust electrotransport conditions to vary the flux of the lisuride or pharmaceutically acceptable lisuride salt, and, thus, maintain plasma concentrations of lisuride or pharmaceutically acceptable lisuride salt at therapeutically desired levels.

A preferred way to delivery treatment for Parkinson's disease is the electrotransport transdermal delivery of cationic lisuride from a donor reservoir containing lisuride or a pharmaceutically acceptable salt thereof in an aqueous composition. Suitable pharmaceutically acceptable salts of lisuride include, without limitation, acetate, propionate, butyrate, pentanoate, hexanoate, heptanoate, levulinate, hydrochloride, hydrobromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate, and sulfonate. An especially useful salt is hydrocholoride, since the chloride ion is present in the body and chloride tends to precipitate out the competing metal ion, such as silver ion, that is generated from the metal donor electrode. Lisuride HCl can be made by, for example, converting from other lisuride salts such as maleate. Lisuride maleate is, of course, commercially available now, since it is used in tablet form for treating Parkinson's Disease already. Lisuride HCl can be obtained from Poli Industria Chimica S.p.A., Via Volturno, 45/48, 20089 Rozzano, Milano, Italy.

The lisuride or pharmaceutically acceptable lisuride salt is present in the donor reservoir in an amount sufficient to deliver the above-described doses transdermally by electrotransport over a desired period of time. The lisuride or pharmaceutically acceptable lisuride salt typically is about 1 wt % to 10 wt % of the donor reservoir formulation (including the weight of the polymeric matrix), more preferably about 1 wt % to 5 wt % of the donor reservoir formulation.

As mentioned above, the administration of lisuride or its salt for treating Parkinson's disease can be done by itself or in conjunction with the administration of levodopa (L-dopa). In such cases, a slightly higher current density, e.g., higher than 50 μA/cm², and larger reservoir area, e.g., 3 cm² or more, can be used. For lisuride monotherapy, in the use of lisuride (or salt) without using levodopa, the rate of lisuride base equivalent administration is about 1 mg/day to 5 mg/day. With a flux of about 5 μg/(cm².hr) to 25 μg/(cm².hr) or more, the area size of the reservoir for lisuride can be about 2 cm² to 10 cm².

In a preferred mode to treating Parkinson's disease, lisuride is co-administered with levodopa. In such a case, levodopa may be administered at a dosage of 300 to 500 mg/day and lisuride may be administered by electrotransport at lisuride free base equivalent of 0.2 mg/day or higher, preferably about 0.5 mg/day to 1.5 mg/day. To this end, lisuride can be administered at free base equivalent of 5 μg/(cm².hr) or more. Such a flux can be achieved by a steady current of about 50 μA/cm² or more. Of course, larger doses can be delivered if needed. The donor reservoir containing lisuride or its salt can contain an aqueous solution of lisuride or solution of a pharmaceutically acceptable salt thereof. As mentioned, lisuride has a net charge of +1 at the acid pH up to about pH7. To provide a composition in the donor reservoir so that lisuride is at the charged state suitable for electrotransport and with less or no irritation to the skin, the pH is preferably about 4-6, more preferably about 5 to 6. The pH can be adjusted by addition of acid or base, or adjusted with ion exchanger before electrotransport. Buffers such as citrate buffer, phosphate buffer, etc. can also be used. Further, peptidic buffer such as that of US Patent Publication No. 20050142531 can be used. In the lisuride donor reservoir, although permeation enhancer (such as esters of fatty acids) can be used, preferably no permeation enhancer is used so as to not include competing ions that may compete with the lisuride cation.

In addition to lisuride and/or its salts, the device is applicable to also delivery other cationic drug (and/or anionic drug) of a wide variety of drugs as long the drug is ionic and can be included in a reservoir to be delivered iontophoretically. Cationic drugs that can be delivered include analgesics, antitumor drugs, antibiotics, histamines, and hormones. Examples of cationic drugs that can be delivered include, e.g., amiloride, digoxin, morphine, procainamide, quinidine, quinine, ranitidine, triamterene, trimethoprim, or vancomycin, procain, lidocaine, dibucaine, morphine, steroids and their salts. For example, hydrochloride salts of vancomycin, procain, lidocaine, dibucaine, and morphine, and acetate salt of medtroxyprogesterone are cationic drugs that can be delivered. Examples of analgesic drug that can be delivered include narcotic analgesic agent and is preferably selected from the group consisting of fentanyl and functional and structural analogs or related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimeheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine. For more effective delivery by electrotransport such as iontophoresis, salts of such analgesic agents are preferably included in the drug reservoir. Suitable salts of cationic drugs, such as lisruide, narcotic analgesic agents, etc., include, without limitation, acetate, propionate, butyrate, pentanoate, hexanoate, heptanoate, levulinate, halides (such as chloride, bromide, iodide), citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. It is known in the art that halide salts are in the form of acid halide for many of such salts (e.g., hydrochloride). The more preferred salt is hydrochloride. Such salts can become ionized in aqueous environment and the cation can be delivered to produce physiological effect on the patient. For example, lisuride maleate or lisuride hydrochloride will provide lisuride cations suitable for iontophoretic delivery.

In additional embodiments, the drug reservoir may optionally contain additional components that can be incorporated into the drug reservoir in any number of ways, i.e., by imbibing the solution into the reservoir matrix, by admixing the drug solution with the matrix material prior to hydrogel formation, or the like. Examples of such additional material can include, e.g., permeation enhancers, stabilizers, dyes, diluents, plasticizer, tackifying agent, pigments, carriers, inert fillers, antioxidants, excipients, gelling agents, anti-irritants, vasoconstrictors, ion exchangers, and other materials as are generally known to the transdermal art. Such materials can be included by on skilled in the art. We have also found that the incorporation of the anion exchanger SEPHADEX™ QAE A-25 (or SEPHADEX™ QAE A-50) in chloride form is especially useful for precipitating silver ions as silver chloride in the reservoir to avoid coloration of the skin by silver. SEPHADEX™ QAE is a quaternary aminoethyl dextran-based resin crosslinked with epichlorohydrin (SEPHADEX™ QAE A-25 and SEPHADEX™ QAE A-50 have quaternary ammonium functionality on a cross-linked dextran supporting carrier structure). SEPHADEX™ ion exchange resins are dextran-based and therefore the monosaccharide in its backbone is glucose. SEPHADEX™ is a dry bead material formed by cross-linking dextran with epichlorohydrin. The SEPHADEX™ QAE A-25 and A-50 are strong anionic exchangers that have about 2.6-3.4 mmol of ionic capacity per gram of dry powder (i.e., with ionic capacity of 2.6-3.4 mmol/g dry basis) and each have particles size range of 40 to 120 microns. The average particle size would be between 40 to 120 microns. The SEPHADEX™ QAE anion exchanger is a strong anion exchanger and has diethyl-(2-hydroxypropyl)aminoethyl functionalities. Strong anion exchangers are resins that remain charged and have high capacity at working pH of 2-12. For weak anion exchangers, not all the anion exchange functionalities are completely ionized at about pH 2-9. Generally, strong anion exchangers are derived from strong bases and weak anion exchangers are derived from weak bases. Tertiary or quaternary ammonium resin can be useful for anion exchange. Quaternary ammonium resins are especially useful for making strong anion exchangers. Strong anion exchangers, e.g., quaternary ammonium resins, are those anion exchangers that are permanently charged under working pH of 2-10, as understood by those skilled in the art. The A-25 has more cross-linking than the A-50 and tends to swell less. The pore size of A-25 has about 30,000 Da exclusion limit and the A-50 has about 200,000 Da exclusion limit. SEPHADEX™ ion exchange resins are available from Sigma-Aldrich in dry powder form commercially (e.g., in 2007 A.D.). A more preferred material is SEPHADEX™ QAE A-25. We have found that SEPHADEX™ is biocompatible that it would not cause irritation and adverse reaction to the skin such as inflammation, erythema or edema. The inclusion of about 5 wt % of SEPHADEX™ ion exchanger is very useful for preventing coloration by metal staining of skin and avoiding irritation. A hydrogel with the anion exchanger can be stacked together with a hydrogel with lisuride to form a reservoir for lisuride electrotransport. The hydrogel layer with the anion exchanger can be on the proximal or distal side of the drug-containing hydrogel (i.e., with lisuride). Such ion exchangers can be used but are not necessary for precipitating out metal ions, e.g., silver ions. If the lisuride salt contains an anion that can precipitate out silver, e.g., lisuride HCl, by including a certain excess amount of the lisuride salt in the donor reservoir, the metal, e.g., silver ions can be precipitated out. Preferably, the device is designed to deliver a predetermined amount, i.e., no more than a maximum amount of drug, such that skin is not discolored by metal staining caused by metal ion migration.

The counter ion reservoir can be made with the same material (e.g., PVOH, etc.) as the donor reservoir and contain a solution of counter ions instead of the drug. For example, in the case the drug is lisuride salt, the counter ion reservoir can contain a sodium salt of the same anion as the lisuride salt. For example, if the lisuride salt is lisuride HCl, the counter ion reservoir can contain saline. A person skilled in the art will know how to make and use the counter ion reservoir and implement the counter ion administration for electrotransport of lisuride in which the donor reservoir contains a lisuride salt.

The eletrotransport devices of the present invention can be included in a kit that contains the device and includes an instruction print, such as an insert or printings on a container, and the like that provides instruction on the how the device is to be applied to a patient and how often the device can be activate and the maximum amount of drug the device is designed to deliver, etc. The instruction of use can include method of activating the device and determining the doses and amount of drug already delivered. The instruction of use can also include brief description of the drug, anion source (e.g., ion exchanger), the construction of the device, the rate of delivery of the drug, pharmacokinetic information, information on disposing the device and warnings.

### Making of the Electrotransport Device

General methods of making gels for reservoirs and incorporating drugs in the gels are known in the art. General methods for making electrodes, printed circuit boards, adhesives, housing, and other kind of iontophoretic device components are known. General methods for making electrotransport devices from their components are known in the art. Generally, components such as the reservoirs, the electrodes, the printed circuit boards, the housing parts, adhesive, displays are made and then the components are assembled by connecting the electrical connections and affixing the separate pieces together for the device to function. For example, the reservoir gel can be laid into a depression in the lower housing to contact the electrode and the lower housing is fitted with the upper housing to enclose the printed circuit between the lower and upper housing. An adhesive, protected by a peelable release liner, is laid on the lower housing to provide adhesion when the device is to be used. The reservoirs can also be made with a hydratable material to form a matrix, such as a hydratable gel, which can be hydrated by infusion with a drug solution before electrotransport. Further, the devices can be made such that different parts can be fitted together right before use. For example, a part of the device can include the electronic control (including the PCB) and another part can include the gels. Before use, the two parts are fitted together allowing electrical control of the delivery of the drug from the donor reservoir.

General methods of using electrotransport devices are known in the art. Generally, a user, such as a patient, more often a care giver (e.g., doctor, nurse, etc.) will open a package pouch, remove the device from the pouch, check the device for proper functioning, remove the peelable protective release liner and apply the device on the body surface of the patient for the device to adhere thereto. During the use period, for a system having structures similar to that in FIG. 1, the control button on the device is manipulated to control the delivery doses of the drug and display of information.

### Treatment of Parkinson's Disease

As mentioned, lisuride can be used to substitute for levodopa in the treatment of Parkinson's disease. In another embodiment, a baseline treatment with lisuride can be administered by other routes, such as by oral administration (e.g., pills, tablets, or liquid, solution), intravenous administration (IV infusion), or by passive transdermal administration. For example, the baseline non-electrotransport lisuride (or a salt) delivery can be from 1 to 4 mg/day lisuride base equivalent. Passive transdermal delivery of lisuride as described in U.S. Pat. No. 5,252,335, can be used. Of course, lisuride tablets are known to those skilled in the art and IV infusion using needles is a well-known technique for drug administration. An electrotransport lisuride delivery device according to the present invention can be used to deliver up to 1.5 mg/day base equivalent of lisuride or a pharmaceutical acceptable salt thereof. Such electrotransport delivery can be done either by a continuous constant DC current over multiple hour period (e.g., 6 hours, 12 hours, 24 hours, etc.) or the device can be initiated by the patient to deliver doses as needed. For example, a dose of a fixed dose size, e.g., about 5 μg to 20 μg can be delivered for a set period of time (e.g., 30 minutes) after each initiation by the patient, e.g., by pushing a button once. The electrotransport device can also be implemented to deliver by pulses or polarity reversal to deliver lisuride.

In one embodiment, no levodopa is used and the entire dosing of lisuride can be done by electrotransport. In such a case, higher flux of lisuride is to be delivered as described above.

Further, lisuride or its salt can be delivered by electrotransport to coadminister with another Parkinson's disease treatment agent, such as another dopamine agonist or levodopa. A baseline administration of levodopa can be delivered to the patient by routes such as IV or oral administration (e.g., pills, tablets, solution). For example, levodopa administration is well known; and a passive transdermal patch for delivery of levodopa is in clinical trial by NeuroDerm, Ltd. Typically, without coadministration with lisuride, for treatment of Parkinson's disease, levodopa is coformulated with carbidopa as a tablet at doses of about 400 to 1600 mg/day (e.g., SINEMET tablets have either 50 mg of carbidopa and 200 mg of levodopa or 25 mg of carbidopa and 100 mg levodopa for sustained release in 4 to 6 hours). Levodopa can be administered without carbidopa, except in that case the bioavailability will be even lower. With a baseline administration of levodopa, an electrotransport lisuride delivery device according to the present invention can be used to deliver up to 1 mg/day base equivalent of lisuride or a pharmaceutical acceptable salt thereof. With the administration of lisuride, it has been reported that the levodopa dosage can be reduced 25%. For example, it has been reported that lisuride administration at about 1.1 mg/day resulted in antiparkinsonian response equal to that achieved with doses of levopoda monotherapy of 668 mg/day. Beneficial effect was also obtained at coadministration with levodopa in which lisuride administration was about 0.8 mg/day (see Movement Disorders, vol 17, Suppl. 4, 2002, p. S74-S78). H. Allain, et al. (Supra) reported an about 1 mg/day of oral lisuride administration along with about 300 to 500 mg/day of levodopa produced beneficial results in Parkinson's disease patients. Thus, lisuride can be delivered via electrotransport either continuously or intermittently as needed by activated by the patient with a baseline delivery of levodopa either at normal levodopa-monotherapy dose or reduced dose. Although other amounts of lisuride can be delivered by electrotransport for treatment of Parkinson's disease, preferably the rate is about 0.5 mg/day to about 5 mg/day. Whether in intermittent delivery or continuous delivery over a period of hours, preferably, the electrotransport device delivers about 0.2 μg/min to 10 μg/min, more preferably about 0.5 to 5 μg/min, even more preferably about 1 to 2 μg/min.

Also, it is noted that other dopamine agonist (other than L-dopa) such as carbidopa (C-dopa), selegiline, rotigotine, etc., may also be used with lisuride for treatment of Parkinson's disease or migraine. If lisuride alone is administered by electrotransport to replace levodopa therapy, it is preferred that the rate of lisuride delivery is not more than 5 mg/day to reduce undesirable side effects.

### EXAMPLES

### Example 1

### Preparation of Hydrogel Reservoir

Hydrogels were typically prepared by dissolving polyvinyl alcohol (PVOH) (MIWOIOL 28-99) at 19 wt % in purified water at 90° C. for 30 minutes. The material was heated until the PVOH went into solution. The temperature was lowered to about 60° C. and maintained for around 30 minutes until solution was free of air bubbles. The resulting mixture was poured into molds and frozen overnight at about −20° C. The PVOH gels were made at 1/32 inch (0.8 mm) (mold thickness). The gels were at an average weight of 0.11 g for 0.03 inch (0.8 mm) molds. The formed hydrogels were then allowed to imbibe a 0.62 wt % aqueous lisuride hydrochloride solution at room temperature to obtain the desired lisuride loading. The drug was imbibed overnight. The gels weighed at an average of 0.18 g after drug imbibing. This resulted in a hydrogel of about 1.2 mm ( 3/64 inch) thick. Alternatively, lisuride loading was achieved by adding lisuride to the PVOH hydrogel solution before freezing. The gels after imbibing were punched to 1.27 cm² circular size and they weighed an average of 0.13 g. With a similar process, another PVOH hydrogel was made by mixing SEPHADEX™ (e.g., QAE-25 available from Sigma-Aldrich) ion exchanger resin in chloride form into PVOH solution in an amount to make a gel of 5 wt % SEPHADEX™ ion exchanger resin and 10 wt % PVOH.

The electrosubstrate (reservoir) was made in the form the dimensions of which are outlined below. The electrosubstrate reservoir had two gel layers for separate polarity, namely cathode and anode. The gel area was maintained at 1.27 cm². The configuration at the anode was made to contain two layers of gel (an SEPHADEX™-containing gel and a drug-containing gel) stacked together. The SEPHADEX™-containing gel was to be positioned closer to the anode electrode and the gel with the drug was placed closer to the skin (named the anode side gel and the skin side gel respectively). Because the two layers are alike in polymeric matrix construction, the two-layered configuration would not affect ion movement adversely. The two layers added up to a thickness of about 2.4 mm.

### Example 2

### In Vitro Experiments of Lisuride Flux

Iontophoretic transdermal flux in vitro measurement using separated human epidermis is well known in the art. The present measurements were made according to such prior known methods. Custom-built DELRON horizontal diffusion cells made in-house were used for all in vitro skin flux experiments. Anode with the same polarity as the drug is adhered to one end of the cell that functions as the donor cell. The counter electrode made of AgCl is adhered at the opposite end. These electrodes are connected to a current generator (Maccor) that applies a direct current across the cell. The Maccor unit is a device with in-built compliance voltage up to 20 V to maintain constant iontophoretic current. For all in vitro electrotransport experiment, heat separated human epidermis was used. In a typical experiment, the epidermis was punched out into suitable circular disks ( 15/16 in, i.e., 2.4 cm diameter) and refrigerated just prior to use. The skin is placed on a screen ( 15/16 in) that fits into the midsection of the DELRON housing assembly. Underneath the screen is a small reservoir that is 0.5 in (1.25 cm) in diameter, 1/16 in (0. 16 cm) deep and can hold approximately 250 μl (microliter) of receptor solution. The stratum corneum side of the skin is placed facing the drug-containing hydrogel. The receptor solution (saline, phosphate or other buffered solutions compatible with the drug) is continuously pumped through the reservoir via polymer tubing (Upchurch Scientific) connected to the end of a syringe/pump assembly. The pump can be set to any desired flow rate. The drug-containing reservoir is placed between the donor electrode and heat separated epidermis.

A custom-built DELRON spacer is used to encase the drug reservoir such that when the entire assembly is assembled together, the drug-containing gel is not pressed against the skin too hard as to puncture it. A number of spacers of varying thickness can be placed together using double-sided adhesives to accommodate polymer films of varying thickness or even multiple films. Double-sided adhesive is used to create a seal between all the DELRON parts and to ensure there are no leaks during the experiment. The entire assembly is placed between two heating blocks that are set at 34° C. to replicate skin temperature. The receptor solution was collected by the collection system, Hanson Research MICROETTE, interfaced to the experimental set up. The samples were collected from the reservoir underneath the skin directly into HPLC vials. The collection system is programmed to collect samples at specified time intervals depending on the length of the experiment, for example, at every hour for 24 hours. The Hanson system is designed such that it can collect from up to twelve cells. From the twelve cells, a piece of tubing takes the receptor solution to the MICROETTE and dispenses it into the HPLC vials loaded onto a rotating wheel that can hold up to 144 vials, or 12 vials for each cell. Once the vials on the wheel are filled, the vials can be replaced with empty vials to collect more samples. The samples can then be analyzed via HPLC to determine delivery efficiency of the drug in the formulation. A 1/10 diluted Delbeccos phosphate buffered saline (DPBS) receptor solution has been used as the receiver fluid in vitro since it showed a good correlation of in vivo in vitro flux in the prior art. The buffer is pumped into the receptor solution reservoir at 1 ml/hr. The Hansen MICROETTE collection system can be programmed to collect periodically, e.g., every 1½ hour for 16 intervals over a 24 hour delivery experiment, or every 45 minutes for 12 hours, etc. The receptor solution flow can also be adjusted to higher or lower values. The cathodic hydrogel was similar to the anodic hydrogel except that it did not contain any drug but saline.

The experiments were done with the double layer stacked reservoirs. Control experiments were done with hydrogels containing fentanyl hydrochloride (fentanyl HCl) in place of lisuride HCl. The reservoir containing fentanyl hydrochloride was made the same way as the lisuride HCl reservoir, except that the fentanyl HCl was 1.05 wt % in the whole reservoir, which was equivalent to 60% of the drug loading of the prior IONSYS™ fentanyl delivery systems, which had 1.74 wt % Fentanyl HCl. It has been shown that 60% fentanyl HCl loading in the reservoir including gel using SEPHADEX™ QAE-25 as being done here would be adequate for electrotrasport and prevent skin silver staining comparable with 1.74 wt % fentanyl HCl loading (i.e., 100% of IONSYS™ loading) with a reservoir without anion exchange resin material.

The anodic electrodes were made with silver and the cathodes were made with silver/silver chloride as known in the art, such as prior art IONSYS™ systems. The current was maintained at 50 μA/cm² for about 12 hours before it was switched off to allow the monitoring to continue for 6 hours. Thus, flux was measured with 12 hours of active electrotransport and 6 hours of passive drug movement.

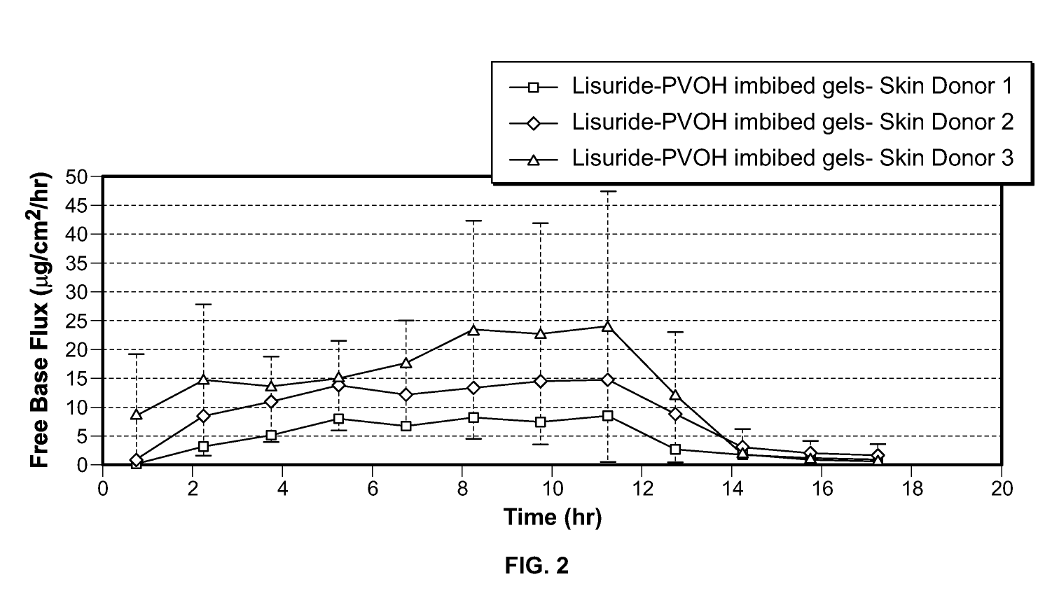

Skin samples from three donors (Donors 1, 2, and 3) were used. FIG. 2 shows the result of the lisuride flux in lisuride base equivalent as a function of time. The bottom curve with square data points represents the averaged data for Donor 1 (3 runs). The middle curve with diamond-shaped data points represents the averaged data for Donor 2 (3 runs). The top curve with triangular data points is the curve of averaged data for Donor 3 (3 runs). The vertical lines associated with the data points represent the standard deviations. The data show that after about 2 hours of electrotransport for two of the skin donors the flux increased to above 5 μg/(cm²hr). After about 4 hours of electrotransport the flux increased to above 5 μg/(cm²hr) for all three of the Donors. Steady state flux was reached at about 8 hours of electrotransport for all three Donors (although for Donors 1 and 2 the fluxes were quite stable in elctrotransport from the fifth hour on). Even the lowest flux (of Donor 1) had a steady state flux of about 8 μg/(cm²hr). After the current was switched off at the 12^{th} hour, the flux fell to about zero at about the 14^{th} hour. Since the data points shown are at 45 min intervals, changes might have happened faster than shown by the curves since they could have happened between the data points. Thus, the data show that electrotransport can be used to delivery lisuride transdermally at a flux of lisuride base equivalent above 5 μg/(cm²hr) and above 8 μg/(cm²hr) with a current of 50 μA/cm².

In the electrotransport flux runs of Donor 2, inadvertently in one of the runs two lisuride-containing layers were stacked together to form the reservoir instead of one lisuride-containing layer and one SEPHADEX™-containing layer. In this run with two lisuride-containing layers, the flux was about twice the flux of the other two runs (which both had steady state flux of about 10 μg/(cm²hr). Thus, the average flux for all three runs was about 15 μg/(cm²hr) at steady state. A possible reason for the higher flux in the run with the double lisuride-containing layers might be that there were relatively fewer competing ions in the system with two drug-containing layers, as well as that a higher drug concentration in the double lisuride-containing system might have an effect on flux. A higher drug concentration might result in a slightly higher passive flux (by transdermal diffusion). Thus, flux of 15 μg/(cm²hr) would be achievable with a current of 50 μA/cm². Since lisuride cation is driven by electrical potential difference and flux would be proportional to current, it is understood by one skilled in the art that even higher flux would be achievable with higher current, e.g., by doubling the current to double the flux.

At the end of the experiments silver chloride was found participated in the skin-side gel with SEPHADEX™ ion exchanger. However, there was no observable silver staining (discoloration) on the skin, except in the experiment run on Donor 2 done without SEPHADEX™ ion exchangers. For all three donors, the pH values were stable for 12 hours. Control experiments were also performed in the flux of fentanyl (the control systems having fentanyl hydrochloride) along side the lisuride experiments to indicate the functioning of the equipment. The control flux of fentanyl in fentanyl base equivalent by electrotransport done in accordance with this invention. The fentanyl runs showed that the flux was good up to about the 11^{th} hour and fell quickly, indicating the depletion of fentanyl HCl in the system. The overall data of the fentanyl controls indicated that equipment was probably functioning for the lisuiride runs, since they were done at the same time. For comparison, the control fentanyl experiments with SEPHADEX™ also showed that there was no observable silver staining for the fentanyl delivery controls. In the control experiments delivering fentanyl through Donor 1 and Donor 2 skin, the flux went to about 8 μg/(cm²hr) at the plateau (steady state) flux.

Thus, the experiments show that lisuride can be delivered by electrotransport for about 12 hours to achieve a steady state flux of about 8 μg/(cm²hr) without silver staining of the skin with a current of 50 μA/cm². Although lisuride HCl was used as the exemplary ingredient for the donor reservoir as the lisuride cation to be delivered by electrotransport, a person skilled in the art will understand that other lisuride salts that can ionize in an aqueous environment can supply lisuride cation to be driven transdermally by iontophoretic means.

The above-described exemplary embodiments are intended to be illustrative in all respects, rather than restrictive, of the present invention. It is to be understood that various combinations and permutations of various parts and components of the schemes disclosed herein can be implemented by one skilled in the art without departing from the scope of the present invention.

We claim:

1. A device for the transdermal electrotransport of lisuride to a patient in need thereof for therapeutic benefit comprising
a donor electrode assembly comprising a donor reservoir that comprises lisuride salt;
a counter electrode assembly; and
controller electrically connected to the donor and counter electrode assemblies for controlling current for the electrotransport of lisuride for therapeutic benefit.

2. The device of claim 1 wherein the lisuride salt is lisuride hydrochloride and the counter electrode assembly contains a counter reservoir having chloride counter ion.

3. The device of claim 2 wherein the controller is operatable to direct a current to administer to the patient at a dose of 0.5 mg/day to 5 mg/day.

4. The device of claim 2 wherein the controller is operatable to direct a current of 50 to 500 μA for electrotransport.

5. The device of claim 2 wherein the device is operatable to deliver by electrotransport at a lisuride base equivalent flux of at least 5 μg/(cm²hr) on donor reservoir surface area.

6. The device of claim 2 wherein the device is operatable to deliver by electrotransport at a lisuride base equivalent flux of 8 μg/(cm²hr) to 50 μg/(cm²hr) on donor reservoir surface area.

7. The device of claim 2 wherein the donor reservoir contains lisuride hydrochloride and the device is operatable to deliver by electrotransport at a lisuride base equivalent flux of 8 μg/(cm²hr) to 50 μg/(cm²hr) on donor reservoir surface area with a current of 50 μA to 100 μA.

8. The device of claim 2 wherein the device is operatable to for delivery for at least 12 hours without staining the skin.

9. The device of claim 2 where the donor electrode assembly contains ion exchanger in chloride form.

10. The device of claim 2 where the donor reservoir contains no permeation enhancer.

11. The device of claim 2 where the donor electrode assembly contains lisuride salt at a pH of 5 to 6.

12. The device of claim 2 wherein the controller is operatable to deliver electrical power as a direct current, a pulsed current, or an alternating reverse polarity current.

13. A method for the transdermal electrotransport of lisuride to a patient in need thereof for therapeutic benefit comprising:
placing a device for the electrotransport delivery of lisuride on a patient, the device comprising
a donor electrode assembly comprising a donor reservoir that comprises lisuride salt;
a counter electrode assembly; and
controller electrically connected to the donor and counter electrode assemblies to control current flow; and
using the device to administer lisuride by electrotransport for therapeutic effect.

14. The method of claim 13 wherein the lisuride salt is lisuride hydrochloride and the counter electrode assembly contains a counter reservoir having chloride counter ion.

15. The method of claim 14 comprising controlling the device to administer to the patient at a dose of 0.5 mg/day to 5 mg/day.

16. The method of claim 14 comprising controlling the device to deliver 50 to 500 μA for electrotransport.

17. The method of claim 14 comprising controlling the device to deliver by electrotransport at a lisuride base equivalent flux of at least 5 μg/(cm²hr) on donor reservoir surface area.

18. The method of claim 14 comprising controlling the device to deliver by electrotransport at a lisuride base equivalent flux of 8 μg/(cm²hr) to 50 μg/(cm²hr) on donor reservoir surface area.

19. The method of claim 14 wherein the donor reservoir contains lisuride hydrochloride and the method comprising controlling the device to deliver by electrotransport at a lisuride base equivalent flux of 8 μg/(cm²hr) to 50 μg/(cm²hr) on donor reservoir surface area with a current of 50 μA to 100 μA.

20. The method of claim 14 comprising using the device for at least 12 hours without staining the skin.

21. The method of claim 14 where the donor electrode assembly contains ion exchanger in chloride form.

22. The method of claim 14 where the donor reservoir contains no permeation enhancer.

23. The method of claim 14 where the donor electrode assembly contains lisuride salt at a pH of 5 to 6.

24. The method of claim 14 comprising controlling the device to deliver electrical power as a direct current, a pulsed current, or an alternating reverse polarity current.

25. A method of making an electrotransport device for administering lisuride transdermally through a body surface of a patient, comprising:
making an anodic reservoir to include 1 wt % to 10 wt % of lisuride salt therein;
providing an anodic assembly, an cathodic assembly, and control circuitry, the anodic assembly having the anodic reservoir having a surface area for transdermal drug delivery;
providing electrical communication between the control circuitry, the anodic assembly and the cathodic assembly; such that the device is enabled to deliver lisuride by electrotransport transdermally from the anodic reservoir at a flux of at least 5 μg/(cm²hr) on donor reservoir surface area for at least 10 hours.

26. A method of electrotransport of lisuride transdermally through a body surface of a patient without discolorizing the body surface, comprising:
placing a device for the electrotransport delivery of lisuride on a patient, the device comprising
a donor electrode assembly comprising lisuride salt and a halide ion source;
a counter electrode assembly; and
controller electrically connected to the donor electrode assembly and counter electrode assemblies to control electrical current flow; and
using the device to administer lisuride by electrotransport for therapeutic effect for at least 10 hours without staining the body surface.

27. A kit for administering a drug by electrotransport transdermally through a body surface of a patient, comprising:
(a) an electrontransport device a donor electrode assembly comprising lisuride salt and a halide ion source, a counter electrode assembly, and controller electrically connected to the donor electrode assembly and counter electrode assemblies to control electrical current flow; and
(b) an instruction print including instruction on electrotransport delivery of lisuride up to a maximum amount, wherein the maximum amount after delivery will not cause skin discolorization.
